(19) United States
(12) Plant Patent Application Publication
(10) Pub. No.: US 2014/0068825 P1
(43) Pub. Date: Mar. 6, 2014

(54) Floribunda rose plant named 'U.F.O.'

(50) ;

(71) Applicant: James W. COINER, Covina, CA (US)

(72) Inventor: James W. COINER, Covina, CA (US)

(21) Appl. No.: 13/573,189

(22) Filed: Aug. 29, 2012

(51) Int. Cl. A01H 5/00

(52) U.S. Cl. PLT/141

(57) ABSTRACT

A new variety of Floribunda rose suitable for garden decoration, having flowers of orange coloration.

### CLASSIFICATION

The present invention relates to a new Rosa hybrida plant.

### VARIETY DENOMINATION

The new plant has the varietal denomination ‘U.F.O.’.

### BACKGROUND OF THE INVENTION

This invention relates to a new and distinct variety of Floribunda Rose. It has as its seed parent the variety known as unknown variety.

### SUMMARY OF THE INVENTION

Among the features which distinguish the new variety from other presently available and commercial rose cultivars known to the inventor is the following combination of characteristics: The plant is a bushy compact plant with upright spreading growth habit, suitable for outdoor garden decoration.

Asexual reproduction of the new variety by budding as performed in Kern County Calif., shows that the foregoing and other distinguishing characteristics come true to form and are established and transmitted through succeeding asexual propagations. ‘U.F.O.’ may be asexually propagated by cuttings, budding and grafting. The budding and grafting successfully occurred on the plant/rootstock Rosa hybrida cv. ‘Dr. Huey’ (unpatented).

### COMPARISON WITH THE CLOSEST COMMERCIALLY AVAILABLE CULTIVAR

The new variety may be distinguished from its closest commercially available cultivar, ‘Easy Living’, by the following combination of characteristics: It has a a unique bloom with flowers of high coloration, with robust vigorous growth habit and bright green foilage.

### BRIEF DESCRIPTION OF ILLUSTRATION

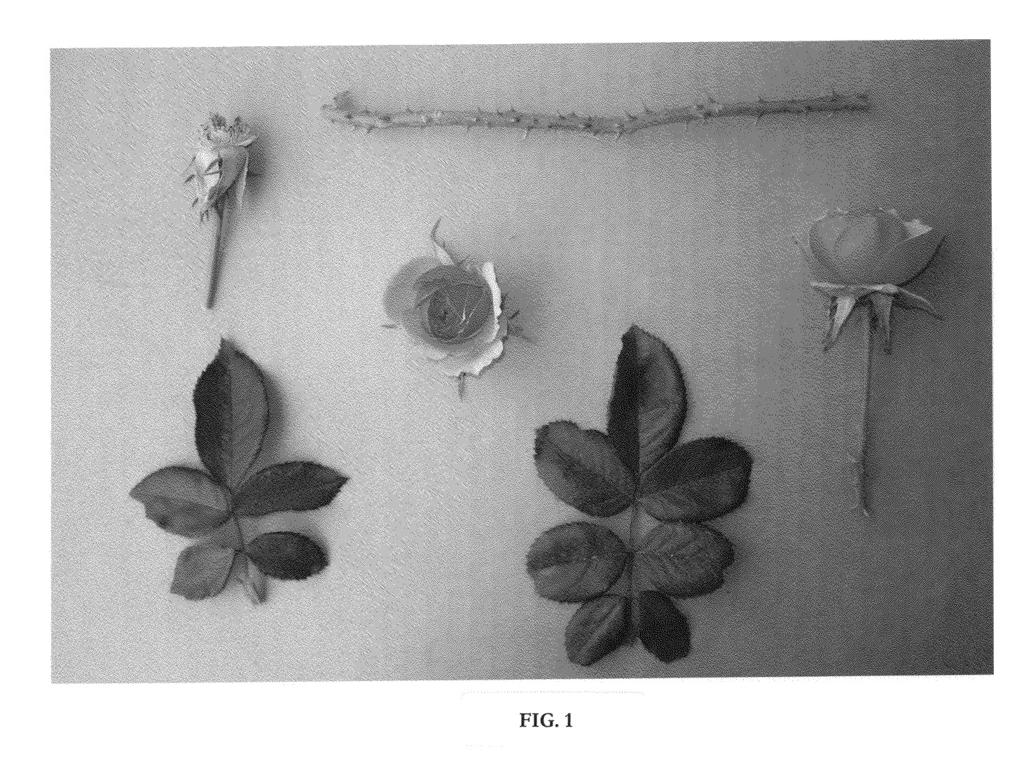

The accompanying photograph, FIG. 1, illustrates the new variety and shows the flowering thereof from bud to full bloom depicted in color as nearly correct as it is possible to make in a color illustration of the character. Throughout this specification, color references and/or values are based upon the Colour Chart of The Royal Horticultural Society (1966) except where common terms of color definition are employed.

### DESCRIPTION OF THE NEW VARIETY

The following description is of two year-old rose plants of the new variety grown outdoors in Wasco, Calif. in the month of October. Phenotypic expression may vary with environmental, cultural and climatic conditions, as well as differences in conditions of light and soil.

### FLOWER

The new variety usually bears its flowers singly, sometimes in clusters of four to five or more per stem. Flowers are borne in regular rounded to pyramidal clusters on strong short to medium stems. Outdoors, the plant blooms abundantly and nearly continuously during the growing season. The flowers have a slight fruity fragrance.

### BUD

The peduncle is about 3.1 cm to about 5.0 cm in length, of slender to average caliper. It is usually smooth, with some stipitate glands, and some hairs and prickles. Peduncle color is near between 144a and 148b. Before the calyx breaks, the bud is about 1.1 cm to about 2.1 cm in diameter at the widest point, about 1.3 cm to about 2.6 cm in length, and ovoid to globular in shape with a conspicuous neck. The surface of the bud bears some foliaceous appendages, stipitate glands and glandular bloom, usually with slender cut bristle-like foliaceous parts extending beyond the tip of the bud about ½ or more of its length. Bud color is near between 144a and 138a.

The sepals are about 2.7 cm to about 4.1 cm in length and about 1 cm to about 1.4 cm in width at the widest point. The outer surface color of the sepal is near between 144a and 138a. The inner surface color of the sepal is near between 137c and 138b and edged with fine wooly tomentum; sepal margins are lined with few stipitate glands and hairs.

The receptacle of the flower is of moderately short to medium length (about 4 cm to about 7 cm) and somewhat thin to average in caliper. The receptacle is pointed to ovoid in form. Its surface is somewhat smooth with moderately thin walls. The receptacle color is near between 144a and 138b.

As the petals open (after the calyx breaks), the bud is about 1.9 cm to about 2.7 cm in diameter at the widest point, about 3.2 cm to about 4.1 cm in length, and very moderately ovoid to globular in form. The color of the under and upper surfaces of the newly opened petals is near between 22b and 24a. The color of the upper surfaces of the newly opened petals is near between 21c and 23b.

### BLOOM

When fully open, the bloom ranges from about 7 cm to about 9 cm in diameter. Petalage is double with about 20 to 26 petals and about 1 to 4 petaloids irregularly arranged. When partially open, the bloom form is moderately cupped to globular, and the petals are somewhat tightly spiraled to cupped with petal edges somewhat rolled inward. When fully open, the bloom form is moderately cupped to full and globular, and the petals are somewhat tightly cupped to imbricated with petal edges moderately rolled outward.

### PETALS

The substance of the petals is somewhat leathery crisp and of medium to thick thickness, with upper surfaces slightly satiny, and under surfaces slightly satiny to velvety. The petals are about 4.1 cm to about 6.2 cm in length and about 4.0 cm. to about 6.1 cm in width at the widest point. Petal margins are somewhat dentate.

The outer petals are very round in shape with apices rounded and usually slightly notched with one to two notches.

The inner petals are very broadly round in shape with apices rounded to flat and usually slightly notched with one to two notches.

Petaloids are about 0.4 cm to about 1 cm in length and about 0.3 cm to about 0.8 cm in width at the widest point. Petaloids are shaped moderately round to cuneate with moderately rounded apices.

### NEWLY OPENED FLOWER

The upper surface color of the outer petals is near between 23c and 23d. The under surface color of the outer petals is near between 32b and 32d.

The upper surface color of the intermediate petals is near between 22b and 24b. The under surface color of the intermediate petals is near between 32b and 33b.

The under and upper surface color of the inner petals is near between 23c and 32d.

The under and upper surface color of the petaloids are similar in coloration to the upper and under surfaces of the intermediate and inner petals.

The general tonality of the newly opened flower is near between 32b and 32d.

### THREE-DAY OLD FLOWER

The upper surface color of the outer petals is near between 23b and 23c. The under surface color of the outer petals is near between 32b and 32d. The under surface color of the inner petals is near between 22b and 24b. The under surface color of the inner petals is near between 32b and 33b.

The under and upper surface color of the petaloids are similar in coloration to the upper and under surfaces of the intermediate and inner petals.

The general tonality of the three-day-old flower is near between 32c and 32d.

On the spent bloom, the petals usually drop off cleanly except for some petaloids that persist.

In October in Wasco, Calif., blooms on the bush growing outdoors generally last about four to five days. Cut roses from plants grown outdoors and kept at normal indoor living temperatures generally last about four to five days.

### MALE REPRODUCTIVE ORGANS

Stamens are average in number (average about 90) and are arranged regularly about the pistils; a few are mixed with petaloids or tucked in the calyx. The filaments are of very moderately medium to long length (about 0.5 cm to about 1.3 cm) most with anthers. Filaments are near between 13b and 12b in color. The anthers are somewhat medium for the class and all open approximately at the same time. Anther color is near between 29a and 30a when immature and near between 178c and 169a at maturity. Pollen is abundant and near between 17c and 15b in color.

### FEMALE REPRODUCTIVE ORGANS

Pistils vary in number (average about 70). The styles are somewhat uneven, moderately average in length (about 0.4 to about 1.1 cm). Stigma color is near between 13c and 12b. Style color is near between 2c and 150d. Ovaries are usually enclosed in the calyx.

Hips have not been observed on this variety when grown in Wasco, Calif.

### FOLIAGE

The compound leaves are usually comprised of three to five leaflets and are borne in normal quantities. The five-leaflet leaves are about 12.2 cm to about 15.6 cm in length and about 10.2 cm to about 12.6 cm in width at the widest point, very moderately crisp in texture and moderately semi-glossy to matte in finish. The terminal leaflets are about 5.4 cm to about 7.9 cm in length and about 3.1 cm to about 4.8 cm in width at the widest point, shaped somewhat obovate with very moderately obtuse to mucronate apices and very moderately obtuse bases. Their margins are somewhat serrate.

The upper surface color of the mature leaf is near between 137a and 137b. The under surface color of the mature leaf is near between 145a and 145b. The upper and under surface color of the young leaf is near between 138a and 138b, sometimes lightly suffused with near between 53a and 53b.

The rachis is somewhat light to average in caliper and somewhat rough. The upper side is shallowly grooved with few hairs and stipitate glands on the edges of the grooves. The under side of the rachis is somewhat rough with few hairs and stipitate glands. The rachis color is near between 178b and 178c.

The stipules are about 1.0 cm to about 1.5 cm in length and somewhat medium width to wide (about 1.0 cm to about 1.5 cm) with moderately short to medium straight points that usually turn out at an angle of less than 45 degrees recurve toward the stem. The stipules color is near between 141c and 141d.

The petiole is moderately light to average in caliper, and somewhat smooth. The upper side is deeply to moderately shallowly grooved with few hairs and stipitate glands. The under side of the rachis is somewhat rough with some hairs and stipitate glands. The petiole color is near between 146a and 146c.

The plant displays an average degree of resistance to powdery mildew and rust as compared to other commercial varieties grown under comparable conditions in Wasco, Calif. The plant's winter hardiness and drought/heat tolerance are yet to be determined.

### GROWTH

The plant has a compact upright medium height growth habit (about 110 cm to about 125 cm in height and about 55 cm to about 38cm cm spread at the widest point), with moderate to full branching. It displays vigorous growth and the canes are of medium caliper for the class.

The color of the major stems is near between 141c and 141d. They bear several small prickles that are about 0.05 cm to about 1.0 cm in length. The prickles are almost straight and angled slightly upward with a very moderately short to medium length with rounded base; prickle color is near between 182b and 182d. The major stem bears many small prickles of similar shape and coloration. There are few fine hairs of similar shape and coloration.

The color of the branches is near between 141 c and 141 d. They bear some prickles which are of similar size and shape to the large prickles on the major stems; prickle color is near between 182b and 182d. There are few hairs of similar shape and coloration which are near between 182b and 182d.

The color of the new shoots is near between 186a and 186b sometimes heavily suffused with near between 190a and 190b. They bear several prickles which are of similar size and shape to the large prickles on the major stems; prickle color is near between 182d and 181d.

I claim:

1. A new and distinct Floribunda rose plant of the variety substantially as described and illustrated herein.
